(19) United States
(12) Patent Application Publication
(10) Pub. No.: US 2012/0272743 A1
(43) Pub. Date: Nov. 1, 2012

(54) Method and Apparatus for Laser-Based Non-Contact Three-Dimensional Borehole Stress Measurement and Pristine Stress Estimation

(76) Inventors: Xiaoqing Sun, Lebanon, NH (US); June Wang, Lebanon, NH (US)

(21) Appl. No.: 13/095,750

(22) Filed: Apr. 27, 2011

(51) Int. Cl. G01N 33/24

(52) U.S. Cl. 73/784

(57) ABSTRACT

A method and apparatus for non-destructively determining borehole stress parameters, that measures acoustic velocities in the rock formation. The apparatus or laser ultrasonic apparatus involving an acoustic signal generator and at least one interferometer sensing unit with shared reference is used to perform non-contact measurement. Horizontal and vertical stresses are evaluated in more than three angular directions (sometimes called azimuths) around the axis of the borehole using acoustoelastic principle. The magnitudes and directions of principal pristine stresses in the rock formation are derived from the measurement data by using closed-form solutions. Magnetometer is used to determine the angular direction of the stress measurement.

### STATEMENT REGARDING FEDERALLY SPONSORED RESEARCH OR DEVELOPMENT

The research involved in this application was funded in part by National Science Foundation, Award ID 1042966. The intellectual property rights of the applicant and the government of the United States of America are governed by 35U.S.C. 202.

### CROSS-REFERENCE TO RELATED APPLICATIONS

Not Applicable.

### REFERENCE TO SEQUENCE LISTING, A TABLE, OR A COMPUTER PROGRAM LISTING COMPACT DISC APPENDIX

Not Applicable.

### BACKGROUND OF THE INVENTION

1. Field of Invention

The present invention relates to in situ stress measurement using optical means, more particularly, to in situ non-contact acoustic velocity measurement using laser ultrasonics, and stress data being obtained through said acoustic velocity based on acoustoelastic theory.

2. Description of the Related Art

Note that the points discussed below may reflect the hindsight gained from the disclosed inventions, and are not necessarily admitted to be prior art.

Crustal stress measurement plays an important role in engineering and scientific problems related to rock faulting, earthquakes, and plate tectonics, as well as in design of oil and gas wells, geothermal reservoirs, underground mines, tunnels, and hydroelectric powerhouses. The introduction of quantitative physical models to explain phenomena in structural geology, tectonics, and seismology, and the development of analytical and numerical methods for the rational design of rock drilling and excavations, frequently require knowledge of the pristine stress regime. It has been recognized that rock stresses cannot be predicted accurately but must be measured. Therefore, a whole new field of study was initiated beginning in the 1950's, dedicated to finding reliable methods of determining stresses in the earth's crust. An accurate in-situ stress measurement will have great influence on the quality, as well as profitability, of many multimillion-dollar research and engineering projects in scientific drilling, petroleum, geothermal, mining and civil engineering. In a report from the National Research Council (Anon., 1994), the development of novel direct sensing of stress is recommended in a long-term effort for the advancement of the nation's drilling and excavation technology in the future.

Since the 1970's, hydrofracturing has dominated stress measurement in deep boreholes. Its simple and rugged equipment and intuitive conceptual reasoning renders the technique particularly suitable for downhole applications. However, experience accumulated from practical use over the years has shown that there are many situations in which this method may not be fully effective or may even fail altogether. Theoretical, experimental and fieldwork have been conducted to address the technical issues related to hydrofracturing. Improvements in hydrofracturing technique over the years generally follow two directions: One is improvement over the conventional approach to hydrofracturing, such as fracture pressurization method or FPM. The other is the development of new stress measurement methods, either by hybrid techniques involving partial use of more than one method or by completely independent approaches such as borehole breakout, borehole slotting, leak-off tests and holographic applications. The general limitations of these downhole stress-monitoring methods are complexity in field applications, slow measurement process, and uncertainty of results. Recently, the hydrofracturing process has been challenged by environmental groups because of possible contamination of underground water resources. Legislation is forthcoming that will mandate more strict EPA regulation of the application of hydrofracturing. If a simple, fast, accurate, non-destructive and environment-friendly method can be developed for downhole in-situ stress measurement, great benefit will be generated not only from research and engineering perspectives, but also for environmental protection.

### BRIEF SUMMARY OF THE INVENTION

It is the object of this innovation to provide a laser-based non-contact apparatus for 3-D stress measurement in downhole applications.

In one embodiment, apparatus of laser ultrasonics is implemented to perform non-contact, non-destructive stress measurement on the internal rock surface of a borehole, based on acoustoelastic theory. The apparatus has no impact on the environment and provides a unique capability for fast and accurate in-situ stress evaluation, meanwhile reducing operating costs associated with stress measurement in the deep earth. Each stress measurement with said apparatus can be completed in the space of several seconds to several minutes, compared to the several hours to several days required by conventional methods.

In one aspect of our embodiment, the device for laser-based 3-D borehole stress measurement is encased in a cylindrical container in order to provide ruggedized protective enclosure for use in the downhole environment.

In another aspect of our embodiment, a pulsed laser beam is projected through a window on said container onto the internal surface of the borehole in order to generate acoustic waves in the wall of the borehole by the thermo-acoustic effect, that is, acoustic waves generated by thermal expansion due to localized heating by the laser beam. Laser interferometer units are used to detect the acoustic signals at specified distances so that the wave velocity of the rock material can be derived in various directions.

In the past, acoustic or seismic wave velocity measurement could be conducted only along the borehole axis. Due to the large size of transducer involved, accurate velocity measurement in a transverse direction was impossible. The laser beams used in this invention can be less than 1 mm in diameter, which makes it easy to obtain accurate wave velocity measurement in transverse direction. This advantage enables the present method of three dimensional borehole stress measurement.

In another embodiment, automatic data processing software is implemented to perform data acquisition and processing online, making simple, fast and accurate downhole stress measurement possible. In one aspect of this embodiment, various mathematical models are innovatively built into said software to allow the determination of the pristine state of the earth stresses.

The disclosed innovation, in various embodiments, provides one or more of the advantages listed below. However, not all of these advantages result from every one of the innovations disclosed, and this list of advantages does not limit the various claimed inventions:
Simple, easy, fast and accurate nondestructive measurement of stresses on borehole surface;
Built-in mathematical modeling for accelerated estimation of pristine stress regime in the surrounding formation; and
Environment friendly.

Other features and advantages will be apparent to those skilled in the art from the following detailed description of the invention.

### BRIEF DESCRIPTION OF THE SEVERAL VIEWS OF THE DRAWING

For a fuller understanding of the nature and object of the present invention, reference is made to the accompanying drawings, wherein:

FIG. 1 shows an example application of the apparatus of the current invention for laser-based non-contact stress measurement in borehole environment.

FIG. 2 shows a block diagram of the hardware configuration of the laser-based stress measurement apparatus in FIG. 1.

FIG. 3 shows software logic chart for the analysis and control of the laser-based stress measurement apparatus in FIG. 1 in accordance with this application.

FIG. 4A shows example operation of the laser-based stress measurement apparatus in FIG. 1.

FIG. 4B shows a simple configuration of the ensonification point and measurement points under Cartesian coordinates.

FIG. 4C shows an example arrangement of measuring points for more precise velocity determination.

FIG. 4D shows example measurements conducted in three separate azimuths for pristine stress regime determination.

FIG. 5A shows an example case for evaluation of ultrasonic velocity of the rock material at zero-stress state by using the laser-based instrumentation in the current invention.

FIG. 5B shows an example case for evaluation of stress-acoustoelastic constant, kᵢᵢ, of the rock samples.

FIG. 5C shows an example case for evaluation of stress-acoustoelastic constant, kᵢⱼ, of the rock samples.

### DETAILED DESCRIPTION OF THE INVENTION

The present application describes several embodiments, and none of the statements below should be taken as limiting the claims generally. The elements in the drawings are not necessarily drawn to scale: some areas or elements may be expanded to help improve understanding of embodiments of the invention. Furthermore, the terms “comprise,” “include,” “have,” and any variations thereof, are intended to cover non-exclusive inclusions, such that a process, method, article, apparatus, or composition that comprises a list of elements is not necessarily limited to those elements, but may include other elements not expressly listed or inherent to such process, method, article, apparatus, or composition.

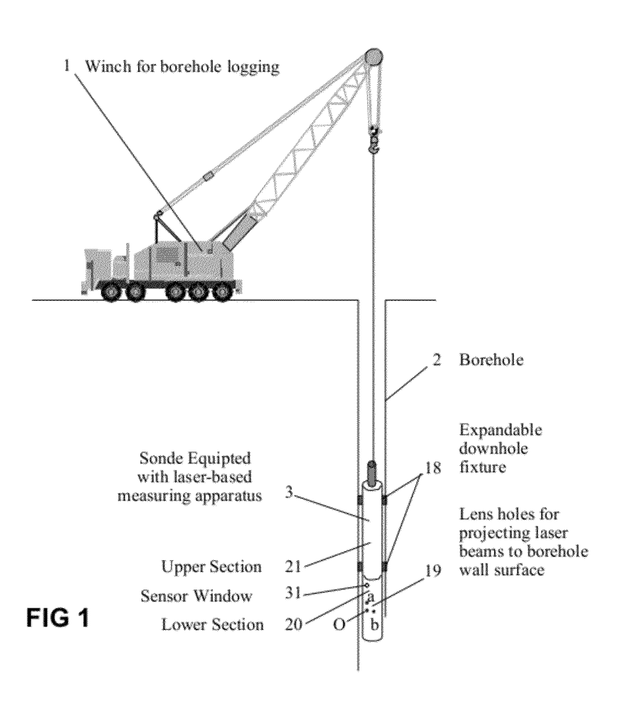

A laser-based non-contact stress measurement in downhole environment involves monitoring of acoustoelastic effect for stress determination. The theory of acoustoelasticity is described in many texts such as Hughes and Kelly (1953). It also takes advantage of recent laser technology for non-contact ultrasonic signal generation and detection. The accompanying software will perform online data processing for in-situ stress assessment, environmental effect compensation, and result presentation. The laser-based non-destructive measuring apparatus may be placed in a sonde or other downhole instrumentation containers with conventional logging equipment such as that shown in FIG. 1, or may be manufactured in an independent unit for downhole stress measurement. In FIG. 1, a sonde 3 equipped with a laser-based stress measuring apparatus is descended in a borehole 2 by winch 1. The sonde 3 is affixed at a certain depth of borehole 2 at its upper section 21 by expandable downhole fixture 18. The stress measuring apparatus is contained in lower section 20 of sonde 3, and performs downhole stress measurement by projecting laser beams to the rock surface through the lens holes 19.

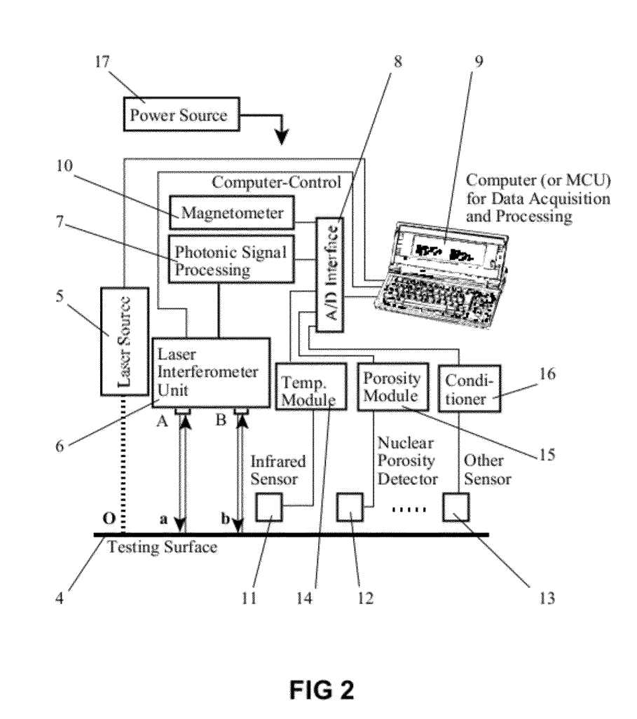

FIG. 2 shows a block diagram of the hardware setup of the invented measuring apparatus. The hardware section performs three major functions: ultrasound injection, signal detection, and environmental parameter collection. The equipment for ultrasound injection is a pulsed laser source 5, e.g. Q-switched Nd:YAG or ruby laser, which creates a thermo-elastic effect on the testing surface 4 (see Point O). In order to ensure optimal system performance, the level of the energy output is adjusted by control from the computer or MCU (microcontroller unit) 9, based on the signals detected by interferometry 6. Signal detection will be accomplished through a laser interferometer 6. Any reference-beam interferometer that can provide sufficient accuracy (such as Michelson, Two-Wave Mixing, Conjugate Mirror, and Photo-Induced Electromotive Force devices) can be used. The detection of ultrasonic signals may be made at two separate locations on the testing surface 4 (Points a and b as shown in FIG. 2) to define wave velocities in two orthogonal directions: the axial direction of the borehole 2 (see FIG. 1) and a tangential direction of the borehole cross-section. The signal detection beams will share the same reference for interference pattern generation so that the output signals from the two measuring points (a and b) are precisely comparable. The photonic signal-processing unit 7 is used to convert the optical phase difference derived from the laser interferometer into proper electric analog signals of ultrasonic displacement at the measuring points for further data processing. Environmental parameters including temperature, porosity, etc. of the testing location will be monitored through various sensing modules in order to compensate for their effects on the subsequent calculations. The examples may include: temperature module 14 and infrared sensor 11 for temperature monitoring, porosity module 15 and nuclear densitometer 12 for porosity, and other sensor(s) 13 with its signal conditioner 16 which is deemed necessary by particular measurement. A magnetometer 10 is also used in this setup to provide direction of the measurement. The detected signals, both ultrasonic and environmental, are then digitized through analog to digital or A/D interface 8 and sent to the computer or MCU 9. The computer or MCU 9 will issue control commands to the hardware setup and will conduct further signal processing to obtain the analytical results of in-situ stress measurement. Power source 17 will supply the necessary electric power for the hardware outlined above.

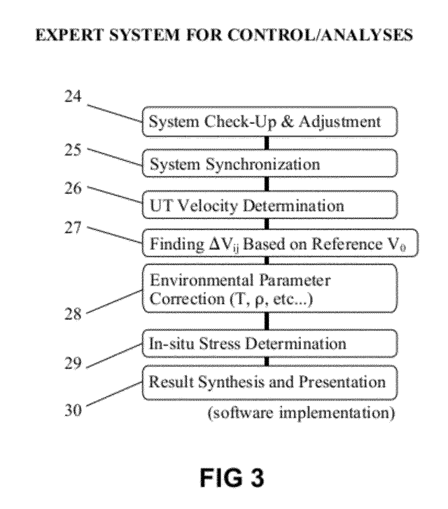

FIG. 3 shows the logic chart for the software section of the laser-based stress-measuring system. This software involves an expert system for control and analyses. It will adjust the hardware for its best performance under different testing conditions and will analyze the data collected to provide results in a format required by the end user. The expert system also maintains a database that stores reference data and physical parameters such as zero-stress wave velocity and thermal expansion coefficient for various rock formations used in the signal analysis. The software includes seven functional modules: System Check-Up & Adjustment 24, System Synchronization 25, UT Velocity Determination 26, Finding ΔVᵢⱼ Based on Reference V₀ 27, Environmental Parameter Correction 28, In-situ Stress Determination 29, and Result Synthesis and Presentation 30.

The first step for the software is to check whether all inputs from the hardware section are at proper levels, and then adjust the output of the pulsed laser source 5 so that the ultrasound signals generated will use the full capacity of the interferometry system, at the same time synchronizing the system so that all parts of the system will work with the same time reference. The expert system will also determine the ultrasonic velocity in both x₁- and x₂-directions, as discussed in the following sections, based on the hardware input. Next, the deviations of ultrasonic velocity from zero-stress state will be determined using the database reference. Then the values of deviations of ultrasonic velocity will be adjusted based on the in-situ environmental parameters. The in-situ normal stress values, σ₁ in x₁-direction and σ₂ in x₂-direction, will then be obtained through the equation of wave velocity and stress in two-dimensional space as shown in EQ 1:

$$\{ \begin{matrix}\frac{{V}_{1} - {V}_{0}}{{V}_{0}} = {k}_{11} {σ}_{1} + {k}_{21} {σ}_{2} \\ \frac{{V}_{2} - {V}_{0}}{{V}_{0}} = {k}_{12} {σ}_{1} + {k}_{22} {σ}_{2}\end{matrix} \qquad EQ 1$$

where V₁ and V₂ are ultrasonic velocities of compressional waves along x₁- and x₂-directions, respectively. V₀ is the zero-stress state reference compressional wave velocity.

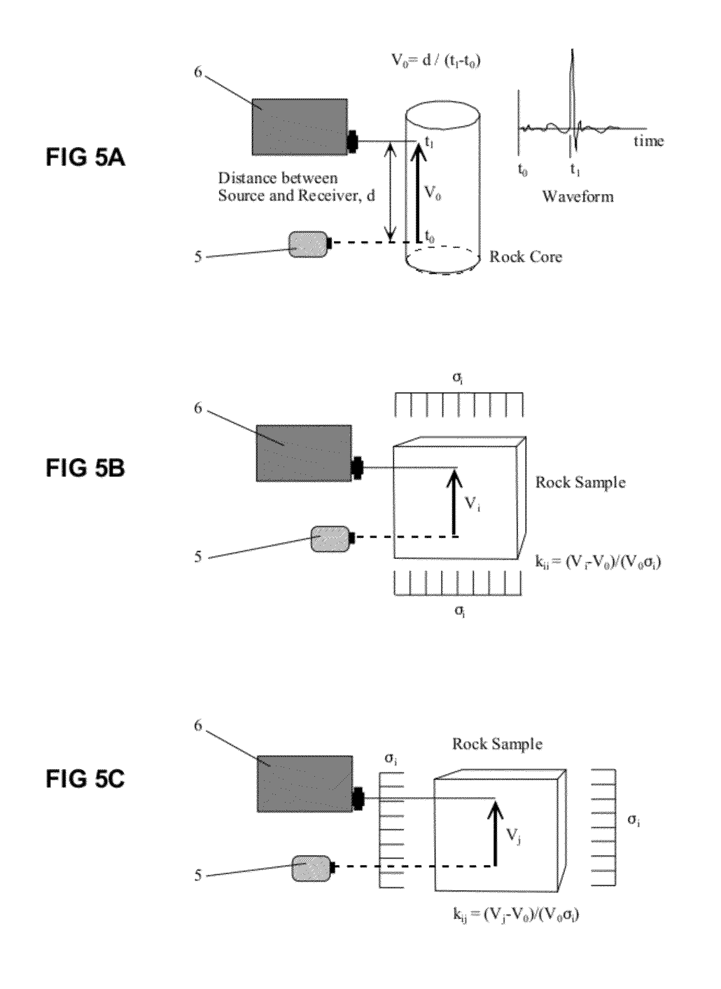

The coefficients kᵢⱼ (i=1, 2; j=1, 2 where i is the direction of stress and j is the direction of its velocity effect) are stress-acoustoelastic constants determined through experiment. Since kᵢⱼ (i=1, 2; j=1, 2) is sensitive to some environmental factors, adjustment may be necessary before its utilization. The said adjustment will be based on a series of tests to evaluate the corresponding stress-acoustoelastic constant kᵢⱼ as shown in FIG. 5B or 5C under three to five variations of the relevant environmental factor, e.g. temperature. A regression, preferably linear regression, of the specific stress-acoustoelastic constants obtained in the tests versus the relevant environmental factor will provide an excellent guide for this adjustment. Finally, the results of stress field description obtained in the borehole measurement will be presented based on the user's requirement.

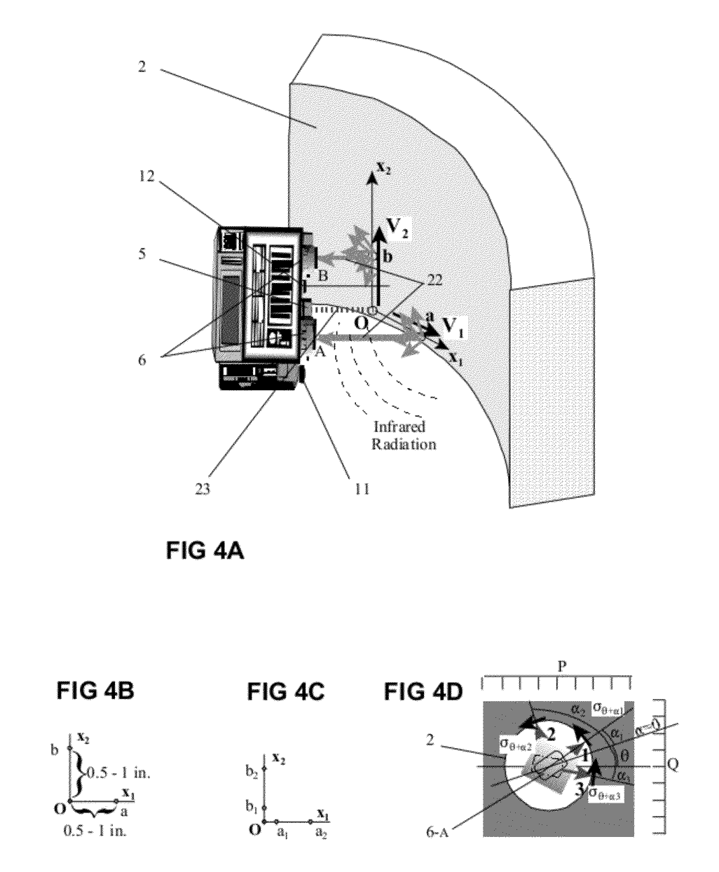

FIG. 4A shows one format of the invention performing downhole stress measurement. The protection shield is removed in this case for a clear view. The pulsed laser beam, as indicated by broken line, is projected onto the wall of borehole 2 at point O for ultrasonic signal generation. The sensing beams A and B for signal detection, as indicated by solid arrow, are projected on points a and b and are intended to measure the wave speed along two orthogonal directions. The direction of Ob is along the axis of the borehole (x₂). Oa is a tangential direction (x₁). The lengths of path Oa and path Ob are about 0.5-1 in. (13-25 mm) as shown in one pattern of measurement in FIG. 4B. The pattern of measuring point distribution may vary. FIG. 4C shows another possible pattern of interrogation that can provide a more precise velocity determination. This is based on the concept of a single transmitter and dual receiver system frequently used in acoustic logging. Strictly speaking, the path Oa is not a straight line but an arc. Since this arc is only a very small portion of the borehole circle, a straight line is used to approximate it. The small amount of error induced by this approximation may be easily corrected in calibration. Then the wave velocity V₁ (along x₁) and V₂ (along x₂) obtained from the measurement will be compared with the sonic velocity of the rock material at zero stress state, V₀, to find velocity change in each direction, and finally the surface stresses will be determined based on EQ 1 by the expert system (software). Thus, by adjusting the elevation and direction of the sonde, accurate vertical and tangential stresses may be obtained at any point on the internal surface along the borehole.

During field measurement, the sonde 3 is lowered down to a certain depth and then anchored to the borehole surface as shown in FIG. 1 by expandable downhole fixture 18, such as expandable claws and pneumatic or hydraulic bladders, in order to provide a stationary platform for the measurement. The sonde 3 consists of two sections: lower section 20 and upper section 21. The upper section is held against the borehole by the fixture 18. The lower section, driven by a stepping motor, is able to rotate around the same axis of the upper section so that various azimuths can be achieved in the measurement. The value of azimuth can be obtained from magnetometer 10. The lens holes 19 on the lower section are used for projecting laser beams onto the rock surface of the wall for measurement: hole O is for pulsed laser of ultrasonic ensonification, holes a and b are for the laser interferometer units. There are also windows 31 for environmental sensors on the lower section 20.

In many cases, the interest is in finding the pristine stress state or the stress state without disturbance caused by the drill hole. Let's establish a polar coordinate system in horizontal plane at the depth of interest, with the origin coincident with the borehole axis. Based on the solution in elasticity, the stress, σ_{θ}, around borehole under polar coordinates can be expressed as (Jaeger and Cook, §10.4. Eq. 15; 1969):

$${σ}_{θ} = \frac{1}{2} ( P + Q ) ( 1 + \frac{{R}^{2}}{{r}^{2}} ) - \frac{1}{2} ( P - Q ) ( 1 + \frac{3 {R}^{4}}{{r}^{4}} ) cos 2 θ \qquad EQ 2$$

where r and θ are indexes of the polar coordinate, R is the radius of the borehole, P and Q are the values of two horizontal principal pristine stresses. Assuming r=R, tangential stress, σ_{θ}, on the internal surface of a borehole can be found by:

σ_{θ}=(P+Q)−2(P−Q) cos 2θ EQ 3

The angle θ of interest is measured from the direction of one principal stress, P. If the stress measurements are conducted at three different locations (azimuths) on the same elevation in a borehole as shown in FIG. 4D, the following equation system may be established based on EQ 3.

$$\{ \begin{matrix}{σ}_{θ + {α}_{1}} = ( P + Q ) - 2 ( P - Q ) cos 2 ( θ + {α}_{1} ) \\ {σ}_{θ + {α}_{2}} = ( P + Q ) - 2 ( P - Q ) cos 2 ( θ + {α}_{2} ) \\ {σ}_{θ + {α}_{3}} = ( P + Q ) - 2 ( P - Q ) cos 2 ( θ + {α}_{3} )\end{matrix} \qquad EQ 4$$

In such a system, α₁, α₂ and α₃ are the direction angles at which the stress measurements are conducted (read from the magnetometer). Angle θ is the angle between the principal stress P and the reference direction of the magnetometer (α=0). The stresses σ_{θ+α1}, σ_{θ+α2}, and σ_{θ+α3} are the horizontal tangential stresses measured at the three locations. The solution of the system (EQ 4) will generate the values of the pristine principal stresses, P and Q, as well as their direction angle θ. The vertical principal pristine stress Z will be an average of the vertical stresses measured at the three locations on the wall. Thus the 3-D pristine stress assessment is completed.

When EQ 4 is used for pristine stress estimate, it is advisable to use special angles for α₁, α₂ and α₃, such as 0, π/4 and π/2, respectively. Although theoretically measurement at any reasonable values of α₁, α₂ and α₃ will provide solution of P, Q and θ, as long as the angles are different from each other, the use of special angles will make the associated mathematics much simpler, as demonstrated below. Suppose α₁=0, α₂=π/4 and α₃=π/2, and let A=P+Q and B=P−Q; EQ 4 becomes:

$$\begin{matrix}{σ}_{θ} = A - 2 B cos 2 θ & EQ 5 \\ {σ}_{θ + \frac{π}{4}} = A + 2 B sin 2 θ & EQ 6 \\ {σ}_{θ + \frac{π}{2}} = A + 2 B cos 2 θ & EQ 7\end{matrix}$$

EQ 5, 6 and 7 are simultaneous equations. EQ 5+EQ 7 yields solution for A:

$$A = \frac{1}{2} ( {σ}_{θ} + {σ}_{θ + \frac{π}{2}} ) \qquad EQ 8$$

Let EQ 7-EQ 5, it is obtained:

$$cos 2 θ = \frac{1}{4 B} ( {σ}_{θ + \frac{π}{2}} - {σ}_{θ} ) or \text{:} {sin}^{2} 2 θ = 1 - {[ \frac{1}{4 B} ( {σ}_{θ + \frac{π}{2}} - {σ}_{θ} ) ]}^{2} \qquad EQ 9$$

By substituting A in EQ 6 with the expression in EQ 8, it is obtained:

$$2 {σ}_{θ + \frac{π}{4}} - {σ}_{θ} - {σ}_{θ + \frac{π}{2}} = 4 B sin 2 θ or \text{:} {sin}^{2} 2 θ = \frac{1}{16 {B}^{2}} {( 2 {σ}_{θ + \frac{π}{4}} - {σ}_{θ} - {σ}_{θ + \frac{π}{2}} )}^{2} \qquad EQ 10$$

Combining EQ 9 and EQ 10, it is obtained:

$$1 - {[ \frac{1}{4 B} ( {σ}_{θ + \frac{π}{2}} - {σ}_{θ} ) ]}^{2} = \frac{1}{16 {B}^{2}} {( 2 {σ}_{θ + \frac{π}{4}} - {σ}_{θ} - {σ}_{θ + \frac{π}{2}} )}^{2} \qquad EQ 11$$

The solution of B is then obtained:

$$B = \frac{1}{4} \sqrt{{( {σ}_{θ + \frac{π}{4}} - {σ}_{θ} )}^{2} + {( 2 {σ}_{θ + \frac{π}{4}} - {σ}_{θ} - {σ}_{θ + \frac{π}{2}} )}^{2}} \qquad EQ 12$$

Once σ_{θ},

$${σ}_{θ + \frac{π}{4}} and {σ}_{θ + \frac{π}{2}}$$

are obtained from the field measurement, the values of A and B are determined. The direction angle θ can be found from either of the EQ 5-7. Because A=P+Q and B=P−Q, the magnitudes of the pristine principal stresses P=(A+B)/2 and Q=(A−B)/2, respectively. The accompanying software will perform all the required calculations.

Calibration is necessary before actual conduct of the stress measurement. Calibration refers to the evaluation of acoustic or sonic velocity, V₀, at zero stress state for each of the rock materials in the formation and the associated stress-acoustoelastic coefficients kᵢⱼ (i=1, 2; j=1, 2). In EQ 1, k₁₂=k₂₁ due to reciprocity. Assuming isotropic material, k₁₁=k₂₂ is obtained. The parameters to be determined for each of the rock materials are V₀, k₁₁ and k₂₁. Three possible practical methods can be used in this context.

1. If rock samples such as cores or lumped debris of sufficient size are available from the formation of interest, the parameters V₀, k₁₁ and k₂₁ can be determined by simple tests in laboratory or in situ as shown in FIGS. 5A, 5B and 5C. Then EQ 1 may be used for evaluation of the stresses σ₁ and σ₂ along the direction of x₁ and x₂ respectively. In the downhole measurement particularly σ₁ and σ₂ can be the tangential (horizontal) stress σ_{θ} and vertical stress a on the internal surface of the borehole respectively at the measurement point.

2. If rock samples are not available, but the zero stress state sonic velocity V₀ may be obtained from earlier geologic records, the following approach may be used.

Let direction x₁ be aligned with tangential (horizontal) direction of the borehole and x₂ be aligned with vertical axis, EQ 1 can be expressed as follows:

$$\{ \begin{matrix}\frac{{V}_{θ}}{{V}_{0}} = 1 + {k}_{11} {σ}_{θ} + {k}_{21} {σ}_{z} \\ \frac{{V}_{z}}{{V}_{0}} = 1 + {k}_{21} {σ}_{θ} + {k}_{11} {σ}_{z}\end{matrix} \qquad EQ 13$$

Substituting the expression in EQ 3 for σ_{θ} in EQ 13, it is obtained that:

$$\{ \begin{matrix}\frac{{V}_{θ}}{{V}_{0}} = 1 + {k}_{11} ( P + Q ) - 2 {k}_{11} ( P - Q ) cos 2 θ + {k}_{21} {σ}_{z} \\ \frac{{V}_{z}}{{V}_{0}} = 1 + {k}_{21} ( P + Q ) - 2 {k}_{21} ( P - Q ) cos 2 θ + {k}_{11} {σ}_{z}\end{matrix} \qquad EQ 14$$

EQ 14 may also be written in the following form:

$$\{ \begin{matrix}\frac{{V}_{θ}}{{V}_{0}} = C + D cos 2 θ \\ \frac{{V}_{z}}{{V}_{0}} = E + F cos 2 θ\end{matrix} \qquad EQ 15$$

where:

C=1+k₁₁(P+Q)+k₂₁σ_{z} EQ 16

D=2k₁₁(P−Q) EQ 17

E=1+k₂₁(P+Q)+k₁₁σ_{z} EQ 18

F=2k₂₁(P−Q) EQ 19

The vertical stress on the borehole surface

$${σ}_{z} = {\sum }_{i} {γ}_{i} {h}_{i}$$

(i=1, 2, . . . ), where γᵢ and hᵢ are the density and thickness of each stratum over the measuring point that are usually known for a given site, which helps to reduce EQ 16-19 to a quaternary form, where the unknowns are k₁₁, k₂₁, P and Q. Since V₀ is known in this case, EQ 15 presents two linear models for V_{θ} and V_{z} as functions of θ, respectively. By conducting in situ measurements using the equipment of the current invention, the measured data [(cos 2θᵢ, V_{θi}/V₀ and V_{zi}/V₀), i=1, 2, . . . ] may be obtained. Then, the coefficients C, D, E and F in EQ 15 may be found by regression of the measurement data against the linear models. Finally, solving the simultaneous equations, EQ 16-19, for the unknowns will generate actual values of k₁₁, k₂₁, P and Q. The values of k₁₁, k₂₁ and the V₀ (known in this case) will then be used for stress evaluation in the latter in-situ stress measurement of the invention.

3. If neither rock samples nor earlier records of sonic velocities are available before the in situ measurement, evaluation of acoustic or sonic velocity, V₀, and the associated coefficients, k₁₁ and k₂₁, may be conducted by first measuring tangential (horizontal) sonic velocity V₁ and vertical velocity V₂ at two depths in a formation with the laser instrumentation in this invention, and then taking stress tests using other technologies such as hydrofracturing to evaluate the in-situ stresses around the borehole at the same depths. Finally, the obtained values of V₁, V₂, σ₁ and σ₂ will be used to find the values of V₀, k₁₁ and k₂₁ through the solution of simultaneous equation system based on the relations defined by EQ 1. After the values of V₀, k₁₁ and k₂₁ are found, the laser-based embodiment will be able to perform downhole stress measurement in a much more effective and economical way, as described above.

The scope of the invention is defined by the appended claims, and includes any changes or modifications to the specific description herein, so long as those changes or modifications remain within the scope of the appended claims.

None of the descriptions in the present application should be read as implying that any particular element, step, or function is an essential element which must be included in the claim scope: the scope of patented subject matter is defined only by the allowed claims.

We claim:

1. A method for non-destructively determining three-dimensional stress parameters of an earth formation at a predetermined depth around a borehole by assessing variations of compressional wave velocities comprising:
a. measuring a first compressional ultrasonic wave velocity in a first direction and a second compressional ultrasonic wave velocity in a second direction orthogonal to said first direction using a non-contact optical means, wherein said first direction and said second direction are preferably horizontal and vertical directions, on the surface of the borehole at said depth in said earth formation;
b. evaluating stress magnitudes in said first direction and said second direction on the internal surface of the borehole in a plurality of azimuths based on acoustoelastic theory using said compressional ultrasonic wave velocities; and
c. calculating principal pristine stress values of said earth formation;
whereby effective, non-contact, non-destructive evaluation of three-dimensional stress parameters can be realized in deep earth formation in downhole environment.

2. The method of claim 1 wherein said non-contact optical means comprising:
a. a pulsed laser source projecting optical beam onto the internal wall of the borehole at an ensonification point, and the optical energy carried by said beam causing localized heating at the ensonification point to generate ultrasonic signals in the rock surface by thermo-expansion effect; and
b. at least one laser interferometer as receiver receiving said ultrasonic signals a distance away from said ensonification point;
whereby the ultrasonic wave velocity in a specific direction defined by said ensonification point and the point of a particular receiver interferometer can be found by dividing the distance between these two points by the first signal arrival time at the particular receiver, commonly recognized as compressional wave velocity in that direction.

3. The method of claim 1 wherein evaluating stress magnitudes in said first direction and said second direction on the internal surface of the borehole may be conducted in the following steps:
a. assigning values to zero stress state wave velocity V₀ and stress-acoustoelastic constants k₁₁ and k₂₁ of the equation of wave velocity and stress in two-dimensional space;
b. solving said equation of wave velocity and stress in two-dimensional space for stress σ₁ in the first direction and stress σ₂ in the second direction in the borehole surface by using the wave velocity V₁ in the first direction and V₂ in the second direction obtained in said measurement with the values of V₀, k₁₁ and k₁₂ found in the previous step; and
c. exercising adjustment to compensate for influence of environmental factors, such as temperature.

4. The method of claim 1 wherein calculating principal pristine stress values of said formation according to the procedures disclosed in the present invention comprising:
a. evaluating magnitudes of horizontal and vertical stresses in the internal surface of a borehole in at least three azimuth directions, preferably with azimuth 0, π/4 and π/2 to a predetermined reference direction;
b. establishing a simultaneous equation system by using the expression of tangential stress σ_{θ} on the internal surface of said borehole and assuming the angle between direction of one principal pristine stress P and said reference direction is θ;
c. determining the magnitudes of horizontal principal pristine stresses P and Q and direction of the principal pristine stress θ by solving said simultaneous equation system; and
d. determining the magnitude of vertical principal pristine stress Z by averaging the vertical stresses obtained at the three azimuths on the borehole surface.

5. An apparatus for non-destructively determining three-dimensional stress parameters of an earth formation at a predetermined depth around a borehole by assessing variations of compressional wave velocities comprising:
a. at least one pulsed laser for non-contact ultrasonic signal generation in internal surface of said borehole;
b. a predetermined number of laser interferometer units preferably sharing one reference beam for non-contact detection of ultrasonic signals in said internal surface of borehole;
c. a magnetometer for determining azimuth of the apparatus during operation;
d. a plurality of sensors for environmental conditions that are considered influential to the testing results and that corrections may be made to the results to compensate for their influences once said environmental conditions are recorded, such as temperature;
e. a computer or microcontroller unit for hardware in test control and signal processing;
f. a storage means for keeping control commands, reference data, and data collected during tests;
g. a communication means, either wired or wireless, to transmit test information from downhole to ground surface and send control commands from ground surface to said testing apparatus;
h. an expert system software for test control and signal processing;
i. a protective enclosure against abuse in downhole environment; and
j. a hoisting means to move said apparatus up and down in the borehole for proper location in measurement operation.

6. The apparatus in claim 5 wherein expert system software comprising the functions of:
a. system check-up and adjustment;
b. system synchronization;
c. ultrasonic velocity determination;
d. finding ultrasonic velocity difference based on the zero stress state velocity;
e. environmental parameter corrections;
f. in-situ stress determination; and
g. result synthesis and presentation.

7. The apparatus in claim 5 wherein said protective enclosure, either being a multipurpose enclosure that also provides protection for other instrumentation or specifically designed for said stress measurement, comprising at least:
a. an upper section with expandable downhole fixture such as expandable claws or pneumatic or hydraulic bladders, whereby the apparatus may be stabilized in a certain location in the borehole; and
b. a lower section attached to said upper section and being able to rotate against the upper section, whereby stress measurement may be conducted in various azimuths.

8. The apparatus in claim 5 wherein a plurality of apertures such as lens holes, sensor windows are embedded in said lower section in certain pattern to expose laser beams and sensors to the internal surface of the borehole for testing, wherein the lens holes are oriented in two orthogonal directions, preferably horizontal and vertical.
